(19) United States
(12) Patent Application Publication
(10) Pub. No.: US 2007/0218515 A1
(43) Pub. Date: Sep. 20, 2007

(54) Method and kits for the determination of the antigens on the red blood cells and antibodies of serum

(76) Inventors: Nina Zhou, Santa Monica, CA (US); Dangyu Hu, New York, NY (US)

Correspondence Address: Nina Zhou, 3 East 101 street, Apartment 4J, New York, NY, 10029, US

(21) Appl. No.: 11/374,557

(22) Filed: Mar. 14, 2006

(51) Int. Cl. G01N 33/00

(52) U.S. Cl. 435/7.25

(57) ABSTRACT

The Present invention relates to biological test methods using micro trays for determining red blood cell antigens and corresponding antibodies of the serum or plasma from individuals using cell aggregation analysis method. This invention also relates to kits for determining red blood cell typing, wherein the kits including mixture monoclonal antibodies, can recognize red blood group's specific antigens. The kits can also have specific red blood cells or cell membrane to determine the corresponding antibodies in the serum. The cell aggregation information may be readable by microscope that may be connected with computer for storage and future analysis. The cell aggregation method in the micro tray can be used for red blood cell antigen determination, antibody recognition and cross match for transfusion with minimal amount of antibody or samples.

### FEDERALLY SPONSORED RESEARCH

Not Applicable

### SEQUENCE LISTING OR PROGRAM

Not Applicable

### BACKGROUND OF THE INVENTION

1. Field of Invention

This invention relates to the field of diagnostic assays and immunohematology analysis of red blood cell typing and antibodies identification present in a biological sample with micro trays.

2. Background of Invention

In 1901 Karl Landsteiner demonstrated that when the blood of one person was transfused with that of another person, differences in his blood typing group might cause of blood transfusion reaction, fever and jaundice. He also classified human blood groups into A, B, and O group and demonstrated that transfusions between humans of the same blood group did not result in the destruction of blood cells. A fourth main blood group-AB was found in 1902 by A. Decastrello and A. Sturli. The Rh blood group system was discovered in 1940 by Levine and Stetson. The term Rh positive and Rh negative now refer only to the presence or absence of one antigen in the system, called Rh₀ or D.

Knowledge of individual blood group antigens is important for blood transfusion. The blood sample can be isolated to plasma and red blood cells. The serum or plasma or blood cells can mix with known typing group of red blood cells or known antibodies of serum or plasma to identify the unknown red blood cell grouping and give the same blood typing blood to eliminate adverse reactions that can occur after blood transfusions.

The structures of the antigen determinants for the red blood cells were established in the 1950s by Watkins and Morgan (Nature 180:1038-1040, 1957). Many antibodies were identified from human being through the isolated known blood grouping serum to use in the blood typing tests. After 1980's, many monoclonal antibodies to antigens on the red blood cells were produced to test red blood cell grouping in the clinic.

The commercial available monoclonal or polyclonal antibody reagents are used to test for the presence of the A, B and D antigens on the surface of erythrocytes. Agglutination with appropriate reagents indicates the presence of that antigen on the surface of the red blood cells tested. Reverse grouping is performed to confirm the presence of expected naturally occurring antibodies in the serum or plasma.

In a conventional blood typing test, drops of typing serum and donor or recipient red blood cells are placed in test tubes, slides, gels, or micro plates and are incubated together. A drop of rabbit anti-IgG antibody (Coomb's reagent) is added to the mixture to induce agglutination between cells and antibodies. This test is known as an indirect agglutination test or an indirect Coomb's test. Today, the most blood banks typically use reagents from manufacture to perform typing. Each of these methods is designed to detect red blood cell antigens using antibodies which have been produced in lymphocyte, either as monoclonal or polyclonal antibodies.

There still remains a need in this field of red cell blood antigen determination, antibody identification for more accurate, efficient, fast, convenient and less expensive technique for typing. As compared to prior methods and systems, Nina Zhou and Dangyu Hu (hereto referred as the “Present Invention”) have developed an accurate, simple, fast, convenient, inexpensive method and typing kits for imaging and analyzing cells and their components. This method and developed kits use commercially available micro trays using microscope to detect cell aggregation for identification of blood typing.

The method Nina Zhou and Dangyu Hu developed with micro trays contains known antigens of fresh or frozen red blood cells or red blood cell membrane which are called screening cells. Each screening cell type or cell membrane can have individual antigenic profile. The combination of the screening cells or cell membrane used must contain antigenic expressions of -C,-c,-E,-e,-K,-k,-Le^{a},-Le^{b},-JK^{a}-JK^{b},-Fy^{a}-Fy^{b},-Pl,-M,-N,-S, and -s homozygous (double dose) for detection of clinically significant antibodies. For example, a red blood cell that is JK (a+b−) is included in the screening cells because antibodies to JK^{a} can show dosage effect. Dosage is demonstrated when antibody reaction is observed on cells that have a double-dose expression of antigen (homozygous) and reaction is weakened or not observed on cells that contain a single dose of antigen (heterozygous).

### SUMMARY OF INVENTION

The Present Invention relates to a method for determining individual blood group antigens. The Present Invention is using micro trays with 60 or 72 wells in which each well was added with one (1) ul monoclonal or polyclonal antibodies to determinate the individual red blood antigens. Combined the variety of antibodies in the wells, the red blood cell antigen phenotype, such as ABO and Rh antigen system, can then be accurately recognized.

The Present Invention relates to a method for determining individual antibodies containing in the serum or plasma. The Present Invention is using micro trays with 60 or 72 wells to store one (1) ul represented screening cells, which can use frozen red blood cells or membranes to determinate the individual red blood cell antibodies. Combined the verity of known antigens on the red blood cells or membranes, the unknown red blood cell antibodies in the serum can be determined.

In the first aspect embodiments, Present Invention is advantageous because it provides a method for testing individual blood group in which the individual person only needs a drop of blood to have his blood typing. That will be very important to the baby, young and old people and patients in the emergence room.

In the second aspect embodiments, the invention provides a method for determining a blood type group of an individual by direct typing on a micro tray comprising known antigens of red blood cells with unknown antibodies of the serum or known antibodies of the serum with unknown blood type group on the red blood cells. Each micro tray has the positive control field and the negative control field as quality control and if the quality control is valid, then it can analyze the aggregation of the test field.

In the third embodiments, the invention provides a method for determining a red blood antigen profile of an individual by direct typing on a micro tray comprising known antibodies with unknown blood type group on the red blood cells. Red blood cell antigens can be an ABO system blood group antigens or an Rh system blood group antigens or an MNSs system blood group antigens or a P system blood group antigens or a Lutheran system blood group antigens or a Kell system blood group antigens or a Lewis system blood group antigens or a Duffy system blood group antigens or a Kidd system blood group antigens or a Fisher system blood group antigens or a red blood cell antigens from any other group.

In the forth embodiments, the invention provides a method for determining a unknown antibodies of the test serum by direct typing on a micro tray comprising known antigens on the red blood cells or cell membranes. In certain embodiments, the red blood cells can have A1 and B cells to identify the ABO system blood group antibodies or the red blood cells can have Rh system blood group antigens to identify Rh antibodies or can have MNSs system blood group antigens to identify MNSs system blood group antibodies or a P system blood group antigens to identify P system antibodies or a Lutheran system blood group antigens to identify Lutheran system blood group or a Kell system blood group antigens to identify Kell group antibodies or a Lewis system blood group antigens to identify Lewis blood antibodies or a Duffy system blood group antigens to identify Duffy blood antibodies or a Kidd system blood group antigens to identify the Kidd blood antibodies a Fisher system blood group antigens to identify Fisher antibodies or other red blood cell antigens to identify corresponded antibodies from any other blood group.

In the fifth embodiments, the first type of immune reaction is a reaction between antibody with the antigens from the red blood cells. After incubation for 15 minutes at 4° C. or room temperature or 37° C., centrifugation and washing, the second type of immune reaction is to add anti-human immunoglobulin, which has an affinity to IgG antibody, in the wells. These two steps will intensify the immune reaction and red blood cell aggregation to identify antigens on the red blood cells and IgG antibodies of the serum.

In the sixth embodiments, when the known antibodies incubate with unknown antigens of red blood cells for antigen detect or known antigens of screening red blood cells with unknown antibodies, some reagents, such as albumin or Albumin, Polyethylene glycol can be equally added in the well to intensify the immunoreactions and cell aggregation.

In the Seventh Invention, the Micro Tray can have Software in the Computer for Analysis of the Aggregation of the Well Profile and Store the Typing Information

In an eighth aspect, the invention provides a method for determining the presence of antibodies to an ABO blood group in an individual's blood sample by reverse-typing on an micro tray including isolated serum from a blood sample; creating at least one (1) sample by mixing serum with cells of a known ABO blood group, such as A1 and B group, or corresponding cell membranes; incubating the sample on the micro tray wells to allow the cells to bind to the antibodies; placing the micro trays to an shaker to mix the cells and antibodies and determining if the red blood cell agglutination under microscope.

According to the above methods, the kits can be produced by selecting monoclonal or polyclonal antibodies against known antigens on the red blood cells into the wells of micro trays for forward typing and the trays have positive and negative controls in the micro trays. These trays can be stored in the minus 70° C. freezer. The trays in this temperature can be stored as long as for five (5) years.

According to the above methods, the kits can be produced by selecting fresh or frozen red blood cells or membranes of known antigens into the wells of micro trays for backward typing and the trays have positive and negative controls in the micro trays. These trays can be stored in the minus 70° C. freezer. The trays in this temperature can be stored as long as for five (5) years.

### DETAILED DESCRIPTION OF THE INVENTION

The invention described herein provides diagnostic assays for red blood cell antigens and antibodies identification from isolated serum or plasma.

Various embodiments of this method of the invention may be similarly designed for the purpose of blood typing, e.g., for testing A, B, 0 system; the Rh system blood group; the MNSs system blood group; the Lutheran system blood group; the Kell system blood group; the Lewis system blood group; the Duffy system blood group; the Kidd system blood group; the Fisher system blood group, or any other blood group. It will be understood by those in the invention field, one or more blood type systems may be simultaneously tested on a single micro tray.

Forward Typing for Antigenic Determinants

Various aspects of the Present Invention are directed to determine the red blood group. The surfaces of matured red blood cells contain large numbers of antigenic determinants that are classified into various blood groups. These antigenic determinants represent red blood cell surface markers that consist of protein and/or carbohydrate moieties. In humans there are at least 23 blood type groups having been reported. The ABO blood grouping is perhaps the most important; serving as the basis for the determination of transfusion compatibility. The Rh group system is strong immunogenic and easy for patient to develop antibody which would cause transfusion reaction.

Thus, individuals whose red blood cells have the A antigen has antibody in his serum directed against the B antigen, and individuals whose red blood cells have the B antigen has antibody in his serum directed against the A antigen. Individuals with both A and B antigens on his red blood cells produces no antibody directed against these A and B antigens, and O typing individuals with neither A and B antigens present on their red blood cells has antibodies directed against both A and B antigens in his serum.

As stated above, the ABO and Rh blood groups are the most important blood cell groups in transfusion medicine. Monoclonal antibodies to the ABO and Rh antigens are commercially available generally made from mice and direct agglutination tests can be performed on red blood cell samples. From the Present Invention, forward typing assays using antibodies against known antigens, e.g. anti-A, anti-B and anti-D monoclonal antibodies in the wells, is a method for determination of the ABO and Rh antigens on the surface of red blood cell.

With respect to the ABO and Rh blood type systems, the forward assay is detected by a known A1, B or AB fresh or frozen blood cells or cell membrane. Antigens present on the surface of the red blood cells are detected by way of a cell immune reaction assay. The other type of blood cell typing assay is referred to add monoclonal or polyclonal antibodies against other antigens, e.g., Kell, Duffy, Kidd, etc. It can also be called as red blood cell phenotypes to detect presence of antigens in individual red blood cells.

The assay is performed within wells of micro trays which can have antibodies against known antigens of red blood cells and mineral oil, for example, test A or B red blood typing, by adding anti-A and anti-B monoclonal antibodies to the wells. The unknown: red blood cells will affix to the wells of micro trays and have immunoreactions with known antibodies in the wells.

Whole blood is first collected, e.g., by finger stick, and the separated red blood cells can be diluted 1 to 3% with normal saline. One (1) ul volume from the diluted red blood cells is then mixed in the well which contains the antibodies. The micro trays then should be put on the shaker to disperse the cells. Also the micro trays can be stirred with stick to mix the cells and antibodies. After the mixture or centrifuge, the trays can be directly read under the microscope.

If antigen phenotype test is performed on the individual blood cells, the red blood cells will be agglutinated as a result of the antibodies bound antigens on the surface of tested red blood cells. For example, the tested red blood cells will mix with the antibodies against known antigen of the red blood cells in the micro wells. After a brief spin of the micro tray, e.g., 300 rpm to 1000 rpm, to set the cells on the well, wash the cells with normal saline and repeat twice centrifugation and wash, then add anti-human immunoglobulin. The micro wells can then be examined under the microscope to determine whether the sample red blood cells are agglutinated.

Some antigens, such as antigens in the Rh system; Kell system; Duffy system; MNSs system; Lewis system; Kidd system and other red blood cell system are tested as no cell aggregation as negative antigens. Whenever these negative results are observed, one (1) ul of control cells weakly sensitized with IgG should be added to make sure the tests are valid.

The known monoclonal or polyclonal antibodies against a number of individual red blood cell antigens are added in each well of micro trays. One (1) micro liter of each antibody with five (5) micro liter mineral oil in the wells is kept in the freezer. After thaw, one (1) micro liter red blood cells from an individual is added in each well of the tray and mixed with antibodies and incubated for 15 minutes at 37° C. The micro trays were then centrifuged at 300 to 1000 rpm for 20 seconds and washed for three (3) times, then two (2) micro liter anti-human globulin are added to the wells. After shaking, the micro trays are examined for agglutination. Agglutination reactions were graded on a scale of 0 to 4+, according to published procedures.

The micro trays can be purchased from commercially available 60 or 72 wells trays. Each well can contain one (1) or two (2) micro liters of the monoclonal or polyclonal antibodies against known antigens or known antigens of fresh red blood cells with glycerol or red cell membranes. After putting the antibodies in the wells, five (5) ul of mineral oil are covered above the antibody or red blood cells or cell membranes, then the tray is covered and stored at minus 20° C. or 70° C. freezer. When shipping to the users, the trays can be thawed in room temperature and kept in the refrigerator for one (1) week for use.

Reverse Typing Assay for Antibody Determination

The invention also provides a method for detecting unknown specific antibodies to an ABO blood group antigen, e.g., assaying a patient's serum for the occurrence of anti-A or anti-B antibodies. In the embodiment, the invention provides a method for reverse typing wherein the sample is loaded onto the micro tray which contains the known antigens of red blood cells, such as A1, B or AB cells or corresponded cell membranes. Fresh or frozen cells can be used.

The Present Invention described herein encompasses two types of blood cell typing. With respect to the ABO blood type systems, the assays include “backward” typing, in which antibodies present in the serum and are detected by way of a cell immune reaction assay. The reverse typing, in which antibodies directed to an ABO phenotype present in a patient's serum, are detected by a known A1, B or AB fresh or frozen blood cells or cell membrane.

As stated above, the ABO and Rh blood groups are the most important groups in transfusion medicine. Known antigens, such as A1 and B red blood cells can be used for ABO and other known antigens of red blood cells can be used for Rh, Duffy, Kell, MNSs, P, Lutheran, Lewis, Kidd systems for antibody detection of serum in the reveres typing. In the method of kits from the Present Invention, backward typing assays are test for antibodies against red blood antigens in the individual serum.

The assay is performed within wells of a micro tray. The wells have known antigens of fresh or frozen red blood cells. A method is described for the determination of the antibodies in the individual serum (e.g., anti-A or anti-B antibody) by the wells of micro trays which stored red blood cells.

The invention also provides a method for detecting in vivo sensitization or coating. It is necessary to test for the presence of incomplete antibodies or complement on the presence of erythrocytes as in cases of suspected hemolytic anemia. This test is the direct antiglobulin test (DAT). Each of the wells from micro tray has been added antiglobulin and mixed with recipient washed red blood cells, and if incomplete antibodies are present, agglutination occurs.

In the first embodiment of reverse typing, whole blood is first collected, e.g., by finger stick, and cells are separated from serum prior to utilizing the serum in the micro tray blood grouping assay. Whole blood can be separated into serum and cell by light centrifugation, for example. The serum, which contains a patient's antibodies, is then mixed with one or more cell types having an ABO cell phenotype. The micro trays which have the serum or plasma are shaken and spin down, then shaken again to disperse the cells. Also the micro trays can be stirred by the needle or stick to mix the cell and serum. After the mixture or centrifuge, the tray can be directly read under microscope.

If antibody screening test is performed in the patient's serum, the red blood cells will be agglutinated as a result of the known antigens on the surface of red blood cells bound with the antibodies of tested serum. For example, the serum will mix with the known antigens of red blood cells in the micro wells. After a brief spin of the micro tray, e.g., 300 rpm to 1000 rpm, to set the cells on the well, wash the cells with normal saline and repeated three time spin and wash, then added anti-human immunoglobulin. The micro wells can then be examined under the microscope to determine whether the sample agglutinated. These antibody determination tests are useful for another unnatural antibodies except for A and B nature antibodies.

Some antibodies, such as antibodies against Rh system; Kell system; Duffy system; MNSs system; Lewis system; Kidd system and other red blood cell system are tested as no cell aggregation as negative antibodies. Whenever these negative results are observed, one (1) ul of control cells weakly sensitized with IgG should be added to make sure the tests are valid.

The known antigens of fresh or frozen red blood cells or cell membranes are added in the wells of the micro trays. One (1) micro liter red blood cells with five (5) micro liter oil are kept in the freezer. After thawing, one (1) micro liter red blood cells from an individual are added in the tray and mixed with antibodies and incubated for 15 minutes at 37° C. The micro trays are then centrifuged at 300 to 1000 rpm for 20 seconds and washed for three (3) times, then two (2) micro liter anti-human globulin are added to the wells. After shaking, the micro trays are examined for agglutination. Agglutination reactions were graded on a scale of 0 to 4+, according to published procedures.

The micro trays can be purchased commercially available 60 or 72 wells trays. Each well can contain known antigens of red blood cells. After putting the serum from individual in the wells, five (5) ul of mineral oil were covered on the antibody or cells, then put the tray covers and stored at minus 20° C. to 70° C. freezer. When shaping to the users, the trays can be thawed in room temperature and kept in the refrigerator for one (1) week for use. The known antigens of red blood cells, which are put into the trays, have to be mix with triglycerol before putting into freezer and also have to be washed for three (3) times before used for antibody typing or screening tests. The washing method can be sucking after spinning down at 300 to 1000 rpm for 20 seconds or just flip down after adding the saline solution.

Software and Related Processing Methods

Information technology resources can be applied to the micro tray in the blood typing techniques. For example, results of the “bar code” and typing results can be viewed under microscope and stored in the computer and analysis by computer software. The typing results and individual “bar code” can be printed out or it can be transmitted (e.g., in secure form) over a network such as the internet for storage or further analysis. The software can be executed, updated, and/or maintained over any professional network.

The method utilizes micro tray with 60 or 72 wells that are conveniently designed into a “bar code” format for sample testing and data presentation. In the forward ABO and RhD blood typing, the micro tray contains three kind of monoclonal antibodies in the wells, which is specific for a particular ABO and D antigenic determinant on the surface of a red blood cell (anti-A, anti-B and anti-D antibodies may be obtained from commercial companies, respectively). In addition, specific immune reaction micro wells are designed as positive (Pos) and negative (Neg) controls. Each “bar code” number will represent each individual typing result.

### EXPERIMENTAL EXAMPLES

### Example 1

Red Blood Cell-A,-B,-D Antigen Typing Assay on Micro Trays

In the following example, the red blood antigen-A,-B,-D typing assay on micro tray assay is conducted on a well of micro trays. The trays can be used from those that are commercially available trays with 60 or 72 wells.

A. Addition of Antibodies Against Known Antigens of Red Blood Cells:

Each one (1) micro liter of anti-A, anti-B and anti-D monoclonal antibodies is added to each of wells in micro tray, then five (5) ul of oil are added to the wells after antibodies were added. The trays are covered and are ready for use. In the same tray, there are positive and negative control wells at first row. At the test time, one (1) micro liter of fresh or frozen known antigen red blood cells, such as A1 and B cells are added in the wells at first row as illustrated in the Table One. The first row will be arranged for quality control with anti-A1, anti-B and anti-D cells and A1 and B cells or cell membrane. That means anti A monoclonal antibody should be positive with A1 cells and negative with B cells and same as anti B monoclonal antibody and anti D monoclonal antibody can not react with Rh D negative red blood cells. The test will be valid only the quality control is working.

b: Prepare Individual Sample.

Whole blood in the anticoagulation (EDTA) from a finger stick is isolated into plasma and red blood cells. 10 ul or 30 ul red blood cells are diluted in 990 to 970 ul of phosphate buffered saline to make a 1 to 3% RBC suspension solution. One (1) ul of red blood cell suspension is added in the wells which contain anti-A, anti-B and anti-D monoclonal antibodies and two (2) ul plasma are added into the micro tray wells which contain A1, B and AB blood group cells with specific syringe (commercial available) and mixed with vortex (commercially available).

c. Identification of Individual Blood Typing

Using a stick or spinning down the micro trays at 300 to 1,000 rpm for 20 seconds to mix the solution in the micro tray wells then vortex again to disperse the cells, the trays can be examined under the microscope to see cell aggregation. After identifying the red blood cell typing, 5% formaldehyde five (5) ul can be added to the wells and the trays can be stored at refrigerator for one month and the results in the tray can be reviewed during such period time.

d. Evaluation:

Agglutination reactions with the MoAbs and individual red blood cells or serum or plasma usually have a 3+ or 4+ score. MoAb anti-A reagents detects all blood type A samples. The anti-A MoAbs dose not react with any blood type B samples, same as anti-B and anti-D. The reverse typing for red blood group A and B is in the same tray with fresh or frozen red blood cells or corresponded cell membrane. The serum or plasma from typing A individual will agglutinate B cells or typing B cell membrane, but not a typing A cells or cell membrane, same as the serum from typing B individual. The blood typing on individual will have no reaction with anti-A and anti-B MoAbs, but his serum or plasma will react with red blood antigen-A,-B and A,B cells or cell membrane. The software of computer is set up such way so that the individual bicode can be scanning in and the typing results can be automatically analyzed and recorded the red blood typing.

TABLE ONE
Red blood cell -A, -B, -D typing

| | 1 | 2 | 3 | 4 | 5 | 6 |
|---|---|---|---|---|---|---|
| | Positive | Positive | Positive | Negative | Negative | Negative |
| | control | control | control | control | control | control |
| A | Anti-A | Anti-B | Anti-D | Anti-A | Anti-B | Anti-D |
| B | Anti-A | Anti-B | Anti-D | A1 Cell | B Cell | A, B Cell |
| C | Anti-A | Anti-B | Anti-D | A1 Cell | B Cell | A, B Cell |
| D | Anti-A | Anti-B | Anti-D | A1 Cell | B Cell | A, B Cell |
| E | Anti-A | Anti-B | Anti-D | A1 Cell | B Cell | A, B Cell |
| F | Anti-A | Anti-B | Anti-D | A1 Cell | B Cell | A, B Cell |
| G | Anti-A | Anti-B | Anti-D | A1 Cell | B Cell | A, B Cell |
| H | Anti-A | Anti-B | Anti-D | A1 Cell | B Cell | A, B Cell |
| I | Anti-A | Anti-B | Anti-D | A1 Cell | B Cell | A, B Cell |
| J | Anti-A | Anti-B | Anti-D | A1 Cell | B Cell | A, B Cell |

### Example 2

Red Blood Cell Phenotype Assay on Micro Trays

In the following example, the red blood cell phenotype assay on micro tray assay is conducted on a well of micro trays. The trays can be commercially available 60 or 70 wells.

a. Addition of Antibody:

The trays for phenotype assay is conducted on a micro tray comprising of: (1) each well containing one (1) ul of specific anti-blood cell group antibodies, such as anti-E,-e,-C,-c,-K,-k,-Fya,-Fyb,-JKa,-JKb,-Lea,-Leb,-M,-N,-S,-s,-P. (2) The each specific monoclonal antibody has duplicate in the wells. (3) five (5) micro liters of mineral oil are added to the each well shown in Table Two.

b. Prepare Individual Sample.

Whole blood in the anticoagulation (EDTA) from a finger stick is isolated into plasma and red blood cells. 10 ul or 30 ul red blood cells are diluted in 990 to 970 ul of phosphate buffered saline to make a 1 to 3% RBC suspension solution. One (1) ul of red blood cell suspension is added in the wells which contain monoclonal antibodies against the surface antigens of red blood cells (commercial available) and mixed with vortex (commercially available).

c. Identification of Individual Blood Phenotype

After mix antibodies with red blood cell suspension, the trays can be put at 37° C. for 15 minutes and after that the trays will be washed with normal saline for three (3) times. After final wash, all of saline solution will be socked out, then two (2) ul of and human IgG will be added each well. The trays will be spin down for 20 second at 300 to 1000 rpm, then vortexes to disperse the cells. To all negative wells, add one (1) ul of control cells weakly sensitized with IgG Negative results can be considered valid if a weakly positive mixed-field reaction is obtained. The trays can be examined under the microscope to see cell aggregation. The identified red blood cell typing results can be import to computer. After identifying the red blood cell phenotype, 5% formaldehyde five (5) ul can be added to the wells and the trays can be stored at refrigerator for one month and the results in the tray can be reviewed during such period time.

d. Evaluation:

Agglutination reactions with the MoAbs and individual red blood cells usually have a 1+ or 3+ score. The Rh; Kell; Duffy, P antigen system will give more strong reaction for red blood cell aggregation, but MNSs, Kidd, Lewis and Lutheran antigen system will give weak reaction for red blood cell aggregation. It should be kept in mind that homozygous (double dose) antigens on the red blood cells will give stronger reaction than heterozygous. Some red blood cell antigens from one individual are richer than the antigens of another individual and also some antibodies are IgG and some are IgM which will affect the cell aggregation extent. Our standard aggregation scales are: 4+ reactions: one large clump, clear background; 3+ reaction: one or two large clumps and several small clumps, clear background and 2+ reaction: Several clumps, clear background. The duplicate well contains same antibodies with same tested red blood cells and should give same results. Computer software can then be set up so that the individual bicode can be scanned and the typing results can be automatically analyzed and red blood typing results recorded.

TABLE TWO
Red blood cell phenotype tray

| | 1 | 2 | 3 | 4 | 5 | 6 |
|---|---|---|---|---|---|---|
| A | Anti-E | Anti-e | Anti-C | Anti-c | Anti-K | Anti-k |
| B | Anti-E | Anti-e | Anti-C | Anti-c | Anti-K | Anti-k |
| C | Anti- | Anti- | Anti- | Anti- | Anti- | Anti- |
| | Fya | Fyb | JKa | JKb | Lea | Leb |
| D | Anti- | Anti- | Anti- | Anti- | Anti- | Anti- |
| | Fya | Fyb | JKa | JKb | Lea | Leb |
| E | Anti-M | Anti-N | Anti-S | Anti-s | Anti-P | |
| F | Anti-M | Anti-N | Anti-S | Anti-s | Anti-P | |

### Example 3

Antibody Screening Typing Assay on Micro Tray.

In the following example, the antibody screening typing assay on micro tray is conducted on a well of micro trays. The trays can be used from commercially available trays with 60 or 72 wells.

a. Addition of one (1) micro liter known antigen screening red blood cell or corresponded membranes into each of wells in micro tray, then five (5) ul of oil are added to the wells after red blood cells were added. The trays are covered and are ready for use. In the same tray, six (6) fresh or frozen red blood cells or corresponded red blood cell membranes in the one row. Each row can test the serum from one individual. One tray with 60 wells can be used for up to 10 individuals and a tray with 70 wells can be used for 12 individuals.

b: Prepare Individual Sample.

Whole blood in the anticoagulation (EDTA) from a finger stick is isolated into plasma and red blood cells. Two (2) ul plasma are added into the micro tray wells which contain screening red blood cells with specific syringe (commercially available) and mixed with vortex (commercially available).

c. Identification Antibodies from Individual Blood Serum.

After mix serum or plasma with red blood cell suspension, the trays can be put at 37° C. for 15 minutes and after that the trays will be washed with normal saline for three (3) times. After final wash, all of saline solution will be socked out, then two (2) ul of and human IgG will be added each well. The trays will be spin down for 20 second at 300 to 1000 rpm, then vortexes to disperse the cells. To all negative wells, add one (1) ul of control cells weakly sensitized with IgG Negative results can be considered valid if a weakly positive mixed-field reaction is obtained. The trays can be examined under the microscope to see cell aggregation. The results of identified antibodies from serum can be import to computer. After identifying the red blood cell phenotype, 5% formaldehyde five (5) ul can be added to the wells and the trays can be stored at refrigerator for one (1) month and the results in the tray can be reviewed during such period time.

d. Evaluation:

Agglutination reactions with the known antigen red blood suspension or cell membranes and antibodies from individual serum usually have a 1+ or 3+ score. The antibodies to Rh; Kell, Duffy and P antigen system will give more strong reaction for red blood cell aggregation, but antibodies to MNSs, Kidd, Lewis and Lutheran antigen system will give weak reaction for red blood cell aggregation. It should be kept in mind that homozygous (double dose) antigens on the red blood cell suspension will give stronger reaction than heterozygous. Some antibodies are IgG and some are IgM which will affect the cell aggregation extent and some antibodies are temperature dependent, so sometimes the tests will be using 4° C. or room temperature except 37° C. detects specific antibodies from individual serum or plasma. Our standard aggregation scales are: 4+ reaction: one large clump, clear background; 3+ reaction: one or two large clumps and several small clumps, clear background; 2+ reaction: Several clumps, clear background. The duplicate well contains same antibodies with same tested red blood cells and should give same results. Computer software can then be set up so that the individual bicode can be scanned and the typing results can be automatically analyzed and red blood typing results recorded.

TABLE THREE
Antibody screening tray

| | | | | | |
|---|---|---|---|---|---|
| Cell I | Cell II | Cell III | Cell IV | Cell V | Cell VI |
| Cell I | Cell II | Cell III | Cell IV | Cell V | Cell VI |
| Cell I | Cell II | Cell III | Cell IV | Cell V | Cell VI |
| Cell I | Cell II | Cell III | Cell IV | Cell V | Cell VI |
| Cell I | Cell II | Cell III | Cell IV | Cell V | Cell VI |
| Cell I | Cell II | Cell III | Cell IV | Cell V | Cell VI |
| Cell I | Cell II | Cell III | Cell IV | Cell V | Cell VI |
| Cell I | Cell II | Cell III | Cell IV | Cell V | Cell VI |
| Cell I | Cell II | Cell III | Cell IV | Cell V | Cell VI |
| Cell I | Cell II | Cell III | Cell IV | Cell V | Cell VI |
| Cell I | Cell II | Cell III | Cell IV | Cell V | Cell VI |

### Example 4

Antibody Determination Test on Micro Tray.

In the following example, the antibody determination typing assay on micro tray is conducted on a well of micro trays. The trays can be from commercially available trays with 60 or 72 wells.

a. Addition of one (1) micro liter known antigen screening red blood cell or corresponded membranes into each of wells in micro tray, then five (5) ul of oil are added to the wells after red blood cells were added. For specific antibodies in the individual serum, the wells will be arranged for three (3) wells with the specific antigen negative and three (3) wells with the specific antigen positive just as Table Four. The antigens range on red blood population will cover 95% of total antigen expression in the red blood cells and have 99% antigens on these red blood cells can be checked out by cell aggregation. The first row has three (3) wells with C antigen negative and three (3) wells with C antigen positive red blood cell suspension. The trays are covered and are ready for use.

b: Prepare Individual Sample.

Whole blood in the anticoagulation (EDTA) from a finger stick is isolated into plasma and red blood cells. Two (2) ul plasma are added into the micro tray wells which contain screening red blood cells with specific syringe (commercially available) and mixed with vortex (commercially available).

c. Identification Antibodies from Individual Blood Serum.

After mix serum or plasma with red blood cell suspension, the trays can be put at 37° C. for 15 minutes and after that the trays will be washed with normal saline for three times. After final wash, all of saline solution will be socked out, then two (2) ul of and human IgG will be added each well. The trays will be spin down for 20 second at 300 to 1000 rpm, then vortexes to disperse the cells. To all negative wells, add one (1) ul of control cells weakly sensitized with IgG Negative results can be considered valid if a weakly positive mixed-field reaction is obtained. The trays can be examined under the microscope to see cell aggregation. The results of identified antibodies from serum can be imported to the computer for analysis. After identifying the red blood cell phenotype, 5% formaldehyde five (5) ul can be added to the wells and the trays can be stored at refrigerator for one (1) month and the results in the tray can be reviewed during such period time.

d. Evaluation:

Agglutination reactions with the known antigen red blood suspension or cell membranes and antibodies from individual serum usually have a 1+ or 3+ score. The antibodies to Rh; Kell, Duffy and P antigen system will give more strong reaction for red blood cell aggregation, but antibodies to MNSs, Kidd, Lewis and Lutheran antigen system will give weak reaction for red blood cell aggregation. It should be kept in mind that homozygous (double dose) antigens on the red blood cell suspension will give stronger reaction than heterozygous. Some antibodies are IgG and some are IgM which will affect the cell aggregation extent and some antibodies are temperature dependent, so sometimes the tests will be using 4° C. or room temperature except 37° C. detects specific antibodies from individual serum or plasma. Our standard aggregation scales are: 4+ reaction: one large clump, clear background; 3+ reaction: one or two large clumps and several small clumps, clear background; 2+ reaction: Several clumps, clear background. The duplicate well contains same antibodies with same tested red blood cells and should give same results. Computer software can then be set up so that the individual bicode can be scanned and the typing results can be automatically analyzed and red blood typing results recorded.

TABLE FOUR
antibody identification

| | | | | | |
|---|---|---|---|---|---|
| C | C | C | C | C | C |
| negative | negative | negative | Positive | Positive | Positive |
| Cell | Cell | Cell | Cell | Cell | Cell |
| C | C | c | c | c | c |
| negative | negative | negative | Positive | Positive | Positive |
| Cell | Cell | Cell | Cell | Cell | Cell |
| E | E | E | E | E | E |
| negative | negative | negative | Positive | Positive | Positive |
| Cell | Cell | Cell | Cell | Cell | Cell |
| E | E | e | e | e | e |
| negative | negative | negative | Positive | Positive | Positive |
| Cell | Cell | Cell | Cell | Cell | Cell |
| Fya | Fya | Fya | Fya | Fya | Fya |
| negative | negative | negative | positive | positive | positive |
| Cell | Cell | Cell | Cell | Cell | Cell |
| Fyb | Fyb | Fyb | Fyb | Fyb | Fyb |
| negative | negative | negative | positive | positive | positive |
| Cell | Cell | Cell | Cell | Cell | Cell |
| K | K | K | K | K | K |
| negative | negative | negative | positive | positive | positive |
| Cell | Cell | Cell | Cell | Cell | Cell |
| K | K | k | k | k | k |
| negative | negative | negative | positive | positive | positive |
| Cell | Cell | Cell | Cell | Cell | Cell |
| M | M | M | M | M | M |
| negative | negative | negative | positive | positive | positive |
| Cell | Cell | Cell | Cell | Cell | Cell |
| N | N | N | N | N | N |
| negative | negative | negative | positive | positive | positive |
| Cell | Cell | Cell | Cell | Cell | Cell |
| S | S | S | S | S | S |
| negative | negative | negative | positive | positive | positive |
| Cell | Cell | Cell | Cell | Cell | Cell |
| S | S | s | s | s | s |
| negative | negative | negative | positive | positive | positive |
| Cell | Cell | Cell | Cell | Cell | Cell |

### Example 5

Cross Match on Micro Tray.

In the following example, the donor red blood cells and recipient serum or plasma are mixed in the micro tray wells. The trays can be from commercially available trays with 60 or 72 wells.

a. Addition of Red Blood Cells and Recipient Serum or Plasma

One (1) micro liter red blood cells from each of donor cells and one (1) micro liter of serum or plasma from recipient is added to each of well of trays with 60 or 70 wells and mixed together.

b: Prepare Individual Sample.

Whole blood in the anticoagulation (EDTA) from a finger stick is isolated into plasma and red blood cells. Two (2) ul plasma are added into the micro tray wells which contain the washed red blood cells from several donor units. If the recipient has some specific antibodies to some antigens of red blood cells, the antigens from donor blood units should be negative for these antibodies.

c. Identification Antibodies from Individual Blood Serum.

After mixing serum or plasma with red blood cell suspension and some reagents, such as albumin, polyethylene glycol(PEG), the trays can be put at 37° C. for 15 minutes and after that the trays can be washed with normal saline for three (3) times. After final wash, all of saline solution need to be socked out, then two (2) ul of and human IgG will be added to each well. The trays will be spin down for 20 seconds at 300 to 1000 rpm, then vortexes to disperse the cells. To all negative wells, add one (1) ul of control cells weakly sensitized with IgG Negative results can be considered valid if a weakly positive mixed-field reaction is obtained. The trays can be examined under the microscope to see cell aggregation. If no aggregation between the red blood cells from donor units with the recipient serum or plasma, the units can be issued for transfusion and red blood cell typing results can be imported to the computer, then the trays can be stored at refrigerator for one week and the results in the tray can be during this period time. After identifying the cross match results, five (5) ul of 5% formaldehyde can be added to the wells and the trays can be stored at refrigerator for one month. The trays could be reviewed upon reaction during transfusion.

d. Evaluation:

Agglutination reaction with the cells from donor units and serum or plasma from recipient usually has no aggregation reaction. The cross match test is very important in the transfusion practice. It can check the recipient and donor unit blood typing and also can check the antibodies in recipient which can not react with the blood from donor. Only the donor blood units which are negative with recipient serum or plasma can be issued for transfusion.

The following is concluded:

A direct benefit of the Present Invention typing approach is that micro volume of blood cells would be used in the analysis of blood samples to identify red blood group and antibody identification. In the example presented, only one (1) ul of anti-A,-B and -D antibodies in wells only need one (1) ul of 1% to 3% of red blood cell suspension for typing. This invention is significantly better than conventional blood typing test which needs at least 20 ul of antibody reagent and 20 ul of typing cells or serum per test.

The Present Invention relates to a method for typing individual blood samples and kits associated therewith. Specifically, the method and kits use a mixture of mono- and poly-clonally antibodies to known antigens or known antigens of red blood cells with tested unknown red blood cells or unknown antibodies of serum to analysis blood groups or antibodies.

The instant invention with a micro tray for typing of red blood cell antigens and antibodies in the plasma is shown and described herein in what is considered to be the most practical, efficient, accurate, and preferred embodiments. This Present Invention speeds the procedure of conventionally procedure with no compromise to test quality and results.

The Present Invention with a micro tray to use one (1) micro liter red blood cells for typing or for antibody identification is shown and described herein in what is considered to be the most economic and convenient embodiments.

The Present invention with a micro tray, microscope and computer to examine the cell aggregation is shown and described herein in what is considered to be the most reliable, accurate and reproducible embodiments.

The kits within the Present Invention method are rapid, simple and accurate and are useful for research and clinical applications, especially in situations where large numbers of blood samples have to be typed quickly and accurately, e.g. blood banks, emergence rooms.

While the Present Invention has been described in detail to a certain preferred embodiments, additional advantages and modifications will be easily apparent to skilled professionals of similar discipline. It should be realized that all of changes, modifications, and variations in the meaning and range of equivalency of the Claims that are noted in this scope below.

What is claimed is:

1. A method for determining a red blood phenotype of an individual by direct typing on a micro tray.

2. The method for determining the presence of antigens to an individual's blood sample phenotype by forward typing on a micro tray with a known blood cell system blood antibodies.

3. The method for determining the presence of antibodies to red blood cells from serum by backward typing on a micro tray with a known blood cell system.

4. The kits which have antibodies in mixture can perform red blood phenotype and recognize blood group system antigens.

5. The kits have known red blood cell antigens for determining the presence of antibodies to red blood cells from serum by backward typing on a micro tray.
